(19) United States
(12) Patent Application Publication
(10) Pub. No.: US 2016/0161490 A1
(43) Pub. Date: Jun. 9, 2016

(54) REAGENT FOR MEASURING ENDOTOXIN AND METHOD FOR MEASURING ENDOTOXIN

(71) Applicant: Sawai Pharmaceutical Co., Ltd., Osaka (JP)

(72) Inventor: Yuu FUJITA, Osaka (JP)

(21) Appl. No.: 14/960,910

(22) Filed: Dec. 7, 2015

(30) Foreign Application Priority Data
Dec. 5, 2014 (JP) 2014-247482

(51) Int. Cl. G01N 33/579

(52) U.S. Cl. CPC G01N 33/579; G01N 2400/50

(57) ABSTRACT

Provided is a method for measuring endotoxin in which glucose-1-phosphate or glucose-6-phosphate is added to a sample and a detection reagent is added to the sample. Furthermore, a reagent for measurement according to the present invention contains glucose-1-phosphate or glucose-6-phosphate. The glucose-1-phosphate or the glucose-6-phosphate may be added in the form of a solution to the sample. The sample may be a pharmaceutical, and a solution containing the glucose-1-phosphate or the glucose-6-phosphate may be prepared so as to have a concentration of the glucose-1-phosphate or the glucose-6-phosphate of 1 mM or more and 25 mM or less.

### CROSS REFERENCE TO RELATED APPLICATIONS

This application is based upon and claims the benefit of priority from the prior Japanese Patent Application No. 2014-247482, filed on Dec. 5, 2014, the entire contents of which are incorporated herein by reference.

### FIELD

The present invention relates to a reagent for measuring endotoxin and a method for measuring endotoxin

### BACKGROUND

Endotoxin is a lipopolysaccharide (LPS), a component of the outer membrane of the cell-wall of a Gram-negative bacterium. Contamination of blood with endotoxin even in trace amounts sometimes causes, for example, a fatal shock, a fever, and disseminated intravascular coagulation (DIC). Therefore, it is important to control endotoxin contamination in pharmaceuticals directly injected into a living body, such as injections, and medical devices. The Japanese Pharmacopoeia specifies Bacterial Endotoxins Test and calls for strict control of endotoxin contamination in pharmaceuticals, such as injections, and medical devices (the 16th edition of the Japanese Pharmacopoeia, the Ministry of Health, Labour and Welfare, Mar. 24, 2011, pp. 79-83). On the other hand, it is known that, in an inflammation resulting from systemic bacterial infection such as sepsis, endotoxin derived from a Gram-negative bacterium, which is one of ausative bacteria, is detected in blood. Blood endotoxin is used as an index for diagnosis of sepsis caused by Gram-negative bacteria.

Endotoxin is present in the outermost part of the outer membrane of the cell-wall of a Gram-negative bacterium, and is roughly divided into a lipid portion that is the innermost part of the endotoxin and called Lipid A, and a sugar chain portion positioned on the outside of the lipid portion. The sugar chain portion comprises a core polysaccharide on a Lipid A side and an O-antigen specific polysaccharide positioned on the outer side. Generally, Lipid A has a β-1,6-diglucosamine skeleton and has a structure in which various fatty acids are combined. It is known that Lipid A is a substance of endotoxin activity.

As for a method for detecting endotoxin in pharmaceuticals, various methods have been conventionally examined. Bacterial Endotoxins Test, which has been currently specified by the Japanese Pharmacopoeia, is a test (limulus test) to detect or quantify bacterial endotoxins of Gram-negative bacterial origin by using a lysate reagent (a limulus reagent) prepared from blood corpuscle extracts of horseshoe crabs (Limulus polyphemus or Tachypleus tridentatus). The Bacterial Endotoxins Test makes use of a coagulation of horseshoe crabs.

The limulus test is a simple and highly-sensitive measurement technique as a system of measuring endotoxin. Therefore, the limulus test has been used not only in the pharmaceutical field but also in a wide range of fields. The principle in the limulus test is that the binding of endotoxin to a factor C contained in a lysate reagent leads to the formation of an activated factor C, whereby a cascade reaction is caused. Coagulin is finally formed and accumulated, whereby white turbidity and gelation are caused. In the limulus test, using such white turbidity and gelation as indexes, endotoxin is detected or endotoxin concentration is calculated. Such limulus test which makes use of the coagulation system of horseshoe crabs can be categorized into several techniques according to differences in detection techniques. For example, there are a gelation technique which makes use of the gel formation of a lysate reagent as an index, and a photometric technique which makes use of an optical change of a lysate reagent as an index. As the photometric technique, there are a turbidimetric technique which makes use of a change in turbidity during the gel formation of a lysate reagent as an index and a chromogenic technique which makes use of color development resulting from hydrolysis of a synthetic substrate contained in a lysate reagent as an index. These detection techniques are listed on the Japanese Pharmacopoeia, and furthermore, have been internationally and widely adopted. (Kenichi Tanamoto, Bulletin of National Institute of Health Sciences 126, pp. 19-33, 2008).

However, the limulus test has also problems. As one of the problems, there has been reported a phenomenon in which a substance contained in a measurement target substance inhibits or enhances the activation of a factor C by endotoxin. This substance is called an interfering factor. When the limulus test is carried out on a pharmaceutical preparation, an additive and an active ingredient themselves contained in the preparation sometimes act as interfering factors. The presence of such interfering factors is one of serious problems in the limulus test. Therefore, the Japanese Pharmacopoeia specifies that, in Bacterial Endotoxins Test, a sample solution is subject to a test for interfering factors to examine the presence of a factor which enhances or inhibits a reaction. In this test for interfering factors, if a sample solution is nonconforming, that is, if an interfering action is observed in the sample solution, measures to eliminate the interfering action from a test solution are needed.

As a measure to eliminate an interfering action from a test solution, a technique to dilute a sample solution has been the most commonly employed. Furthermore, there have been reported, for example, a technique to neutralize an interfering factor by using a buffer solution (Yuu Fujita, et al., Analytical Biochemistry 409, pp. 46-53, 2011) and a technique to neutralize an interfering factor by using a low-concentration surface active agent. However, these techniques have no neutralizing effect on some interfering factors. Furthermore, a surface active agent itself is a substance which affects the activity of endotoxin, and hence, it is hard to say that the technique to use a surface active agent is entirely satisfactory. Furthermore, in the technique to use a buffer solution, nonspecific turbidity is caused by the buffer solution, whereby it is sometimes difficult to carry out an exact measurement of endotoxin, and hence, again, it is hard to say that the technique is entirely satisfactory.

On the other hand, also in the case where the concentration of endotoxin in blood is measured as an inspection item for sepsis diagnosis, the limulus test has been adopted. Also in measurements of the concentration of endotoxin in blood, an interfering action is a serious problem. In plasma, which is a main measurement target substance in inspections for sepsis diagnosis, various proteins are present. Sometimes, endotoxin specifically or nonspecifically binds to proteins in plasma, whereby a limulus reaction is enhanced or inhibited. Currently, as a countermeasure against such interfering action of proteins in plasma, there has been widely employed a dilution and heating technique (Junkichi Takahashi, Endotoxin-kenkyu 12, pp. 113-118, 2009) in which a collected plasma sample is diluted 10 times with water for injection or water for injection which contains a low-concentration surface active agent and the resulting diluent is heated at 70 to 80° C. for 10 minutes, and then added to a lysate reagent. However, even in the case of using this dilution and heating technique, it is difficult to sufficiently neutralize an interfering action, and there is a possibility of forming a false negative diagnosis under the influence of the interfering action. Therefore, in the conventional measurements of the concentration of endotoxin in blood, there is yet room for improvement.

### SUMMARY

One embodiment of the present invention provides a method for measuring endotoxin, wherein glucose-1-phosphate or glucose-6-phosphate is added to a sample, and a detection reagent is added thereto.

In the method for measuring endotoxin, glucose-1-phosphate or glucose-6-phosphate may be added in the form of a solution to the sample.

In the method for measuring endotoxin, the sample may be a non-blood sample.

In the method for measuring endotoxin, the non-blood sample may be a pharmaceutical.

In the method for measuring endotoxin, the pharmaceutical is preferably a pharmaceutical preparation, a drug substance, or an inactive additive.

In the method for measuring endotoxin, the non-blood sample may be a chemical, a reagent, or a medium.

In the method for measuring endotoxin in the non-blood sample, a solution containing the glucose-1-phosphate or the glucose-6-phosphate preferably has a concentration of the glucose-1-phosphate or the glucose-6-phosphate of 1 mM or more and 25 mM or less.

In the method for measuring endotoxin, the sample may be a blood sample.

In the method for measuring endotoxin, the blood sample may be plasma.

In the method for measuring endotoxin, the plasma to which the glucose-1-phosphate or the glucose-6-phosphate is added may be heat-treated, and a detection reagent may be added to heat-treated plasma.

In the method for measuring endotoxin in the blood sample, a solution containing the glucose-1-phosphate or the glucose-6-phosphate preferably has a concentration of the glucose-1-phosphate or the glucose-6-phosphate of 1 mM or more and 5 mM or less.

Furthermore, one embodiment of the present invention provides a reagent for measuring endotoxin, wherein the reagent contains glucose-1-phosphate or glucose-6-phosphate.

The reagent for measuring endotoxin is preferably a solution containing the glucose-1-phosphate or the glucose-6-phosphate.

In the case of using a non-blood sample as a sample, the reagent for measuring endotoxin preferably is a solution having a concentration of the glucose-1-phosphate or the glucose-6-phosphate of 1 mM or more and 25 mM or less.

In the case of using a blood sample as a sample, the reagent for measuring endotoxin preferably is a solution having a concentration of the glucose-1-phosphate or the glucose-6-phosphate of 1 mM or more and 5 mM or less.

### BRIEF DESCRIPTION OF DRAWINGS

FIG. 1 shows the results of endotoxin measurements according to Examples of the present invention;

FIG. 2 shows the results of measurements of endotoxin in rat plasma according to Examples of the present invention;

FIG. 3 shows the results of measurements of endotoxin in human plasma according to Examples of the present invention;

FIG. 4 shows the results of measurements of endotoxin in the plasma of sepsis model rats according to Examples of the present invention; and

FIG. 5 shows the result of a comparison between the concentration of endotoxin in the plasma of sepsis model rats measured using a reagent for measurement according to Examples of the present invention and the concentration of endotoxin in the plasma of the sepsis model rats measured using water for injection.

### DESCRIPTION OF EMBODIMENTS

Hereinafter, the reagent for measuring endotoxin and the method for measuring endotoxin according to the present invention are described. However, the reagent for measuring endotoxin and the method for measuring endotoxin according to the present invention shall not be limited to descriptions of Embodiments and Examples illustrated below.

One of action mechanisms of interference in the limulus test is a mechanism in which an interfering factor directly acts on endotoxin. As such interfering factor, for example, iron sulfate, ciprofloxacin, and epirubicin are known. The inventors conducted earnest studies on a technique for reducing the influence of such interfering factors which directly act on endotoxin, and the inventors focused on a structure of Lipid A, which is a substance of endotoxin activity. As a result of the studies, the inventors found out that these interfering factors cut a glycosidic linkage and an ester linkage of Lipid A via a phosphate group of Lipid A, which is an active center of endotoxin, thereby exerting an interfering action, for example, reduction in the activity of endotoxin (Y. Fujita and T. Nabetani, Journal of Applied Microbiology 116, pp. 89-99, 2014). Lipid A of a colon bacillus (E. coli) is illustrated in Structural Formula (1).

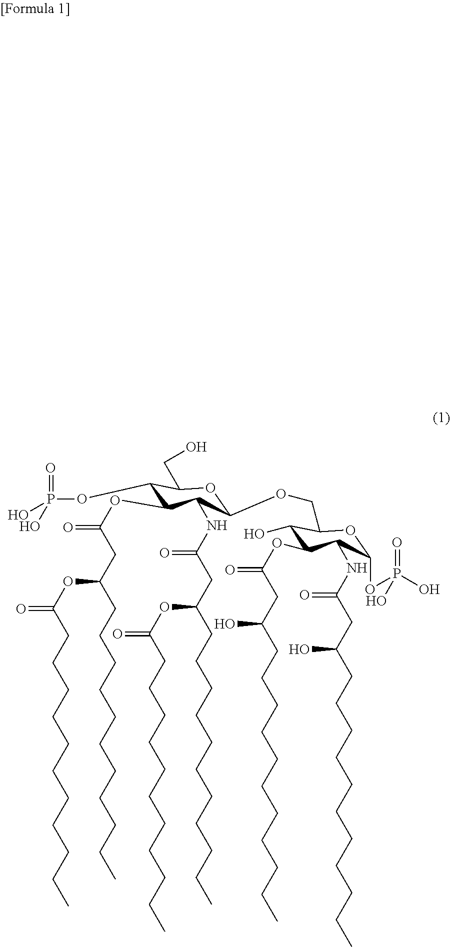

The inventors were the first to find out that the use of glucose-1-phosphate (Structural Formula 2) and glucose-6-phosphate (Structural Formula 3), which are compounds having a structure similar to a phosphorylated β-1,6-diglucosamine skeleton of Lipid A, enables a reduction in the influence of an interfering factor. Previously, such findings have not been reported.

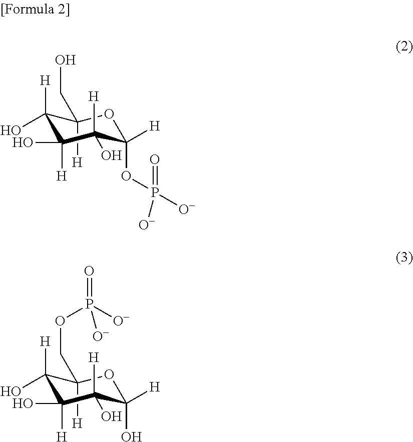

The reagent for measuring endotoxin according to the present invention contains glucose-1-phosphate or glucose-6-phosphate. The reagent for measuring endotoxin according to the present invention is preferably a solution containing glucose-1-phosphate or glucose-6-phosphate. As a solvent of the solution, a solvent which does not affect the activity of endotoxin is preferably employed, and examples of the solvent include endotoxin-free water for injection. Furthermore, a common buffer solution may be used within the bounds of not affecting the activity of endotoxin. Examples of the buffer solution include phosphate buffers, Tris buffers, and BES buffers, but, the buffer solution is not limited to these.

Examples of a sample in a method for measuring endotoxin by using the reagent for measuring endotoxin according to the present invention include non-blood samples, such as pharmaceuticals, and blood samples, such as serum and plasma. Examples of the pharmaceuticals include a pharmaceutical preparation, a drug substance, and an inactive additive. Examples of the pharmaceutical preparation particularly include injections. It should be noted that, in the specification, examples of the pharmaceutical preparations also include an albumin preparation, an immunoglobulin preparation, and a blood coagulation factor preparation. Furthermore, besides the pharmaceuticals, examples of the non-blood sample may include a chemical, a reagent, and a medium. It should be noted that, in the specification, the chemical, the reagent, and the medium may contain a living-body-derived substance, such as albumin, peptone, or amino acid. The sample is not limited to the above-mentioned examples, and, besides the examples, what is strictly required not to be contaminated with endotoxin, such as endotoxin-free water, a medical instrument, or a laboratory instrument, may be used as the sample.

(Method for Measuring Endotoxin in Non-Blood Sample)

A method for measuring endotoxin in a non-blood sample, particularly in a pharmaceutical, by using the reagent for measuring endotoxin according to the present invention will be described. Pharmaceuticals can be in various states, such as solid, semisolid, and liquid states. In the case where endotoxin in a liquid pharmaceutical is measured, the liquid pharmaceutical may be used as it is as a sample, or the liquid pharmaceutical may be diluted with, for example, water for injection to prepare a sample is adjusted. Furthermore, in the case where endotoxin in a semisolid or solid pharmaceutical is measured, a sample may be prepared by making the semisolid or solid pharmaceutical in a state suitable for endotoxin measurement with a well-known technique, such as dissolution or suspension in water for injection or the like.

The reagent for measuring endotoxin according to the present invention preferably is a solution having a concentration of glucose-1-phosphate or glucose-6-phosphate of 1 mM or more and 25 mM or less. When the concentration is lower than 1 mM, the influence of an interfering factor cannot be sufficiently reduced. On the other hand, the concentration is higher than 25 mM, there is a risk that glucose-1-phosphate or glucose-6-phosphate will act on a factor C, thereby affecting the result of measurements of endotoxin. In the case of measuring endotoxin in a non-blood sample, the reagent for measuring endotoxin according to the present invention preferably is a solution having a concentration of glucose-1-phosphate or glucose-6-phosphate of 5 mM.

Glucose-1-phosphate or glucose-6-phosphate is dissolved in, for example, water for injection so as to prepare the reagent for measurement according to the present invention which is a solution having a concentration of glucose-1-phosphate or glucose-6-phosphate of 1 mM or more and 25 mM or less. A sample is diluted with the solution of the reagent for measurement according to the present invention. Alternatively, after the addition of the reagent for measurement to a sample, the sample may be further diluted with, for example, water for injection. Alternatively, after dilution of a sample with water for injection or the like, the reagent for measurement may be added thereto. The dilution ratio of a sample is not particularly limited, and may be arbitrarily set with consideration of the detection limit of the limulus test. For example, a 2-fold or more and 10000-fold or less dilution of a sample is preferable.

A detection reagent is added to a sample to which the reagent for measurement has been added. As the detection reagent, a well-known lysate reagent of Limulus polyphemus or Tachypleus tridentatus origin for limulus tests may be employed. Furthermore, as a lysate reagent, for example, a factor C of recombinant may be employed. After the addition of the detection reagent, incubation is performed at 37° C. for a predetermined time, and the light transmissivity of a reaction solution is measured. For the measurement of light transmissivity and the calculation of endotoxin, well-known techniques may be employed.

For the measurement of endotoxin in a non-blood sample, there may be employed a method which conforms to Bacterial Endotoxins Test specified in the Japanese Pharmacopoeia. It should be noted that the method for measuring endotoxin according to the present invention may be executed by using reagent for measuring endotoxin according to the present invention in combination with, for example, a commercial kit for endotoxin measurement.

In the present invention, the addition of a glucose-1-phosphate-containing or glucose-6-phosphate-containing reagent for measurement to a sample makes it possible to prevent an interfering factor from affecting the activity of endotoxin. In the reagent for measuring endotoxin according to the present invention, a portion in which a phosphate group is added to a sugar of glucose-1-phosphate or glucose-6-phosphate is structurally similar to Lipid A. Hence, it is inferred that glucose-1-phosphate or glucose-6-phosphate competes with a phosphate group of Lipid A for an interfering factor, thereby directly inhibiting interference. Therefore, it is possible to detect endotoxin with higher sensitivity and quantify with higher accuracy than was previously possible.

(Method for Measuring Endotoxin in Blood Sample)

A method for measuring endotoxin in a blood sample, particularly in blood, by using the reagent for measuring endotoxin according to the present invention will be described. In the case where endotoxin in blood is measured, serum or plasma may be used as a sample, but, plasma is preferably used as a sample. The reagent for measuring endotoxin according to the present invention preferably is a solution having a concentration of glucose-1-phosphate or glucose-6-phosphate of 1 mM or more and 5 mM or less. When the concentration is lower than 1 mM, the influence of an interfering factor cannot be sufficiently reduced. On the other hand, the concentration is higher than 5 mM, there is a risk that glucose-1-phosphate or glucose-6-phosphate may cause the precipitation of proteins in plasma, and white turbidity resulting from the precipitation may affect the result of endotoxin measurements. In the case of measuring endotoxin in plasma, the reagent for measuring endotoxin according to the present invention preferably is a solution having a concentration of glucose-1-phosphate or glucose-6-phosphate of 2.5 mM.

Glucose-1-phosphate or glucose-6-phosphate is dissolved in, for example, water for injection so as to prepare the reagent for measurement according to the present invention which is a solution having a concentration of glucose-1-phosphate or glucose-6-phosphate of 1 mM or more and 5 mM or less. A sample is diluted with the solution of the reagent for measurement according to the present invention. Alternatively, after addition of the reagent for measurement to a sample, the sample may be further diluted with, for example, water for injection. Alternatively, after dilution of a sample with, for example, water for injection, the reagent for measurement may be added thereto. The dilution ratio of a sample is not particularly limited, and may be arbitrarily set with consideration of the detection limit of the limulus test. For example, a 10-fold or more and 10000-fold or less dilution of a sample is preferable.

Furthermore, heat treatment of the sample to which the reagent for measurement has been added makes it possible to improve accuracy in endotoxin measurement. For example, in the case where human plasma is used as a sample, the sample is preferably heated at 70 to 80° C. for 10 minutes.

A detection reagent is added to the sample to which the reagent for measurement has been added. As the detection reagent, a well-known lysate reagent of Limulus polyphemus or Tachypleus tridentatus origin for limulus tests may be employed. Furthermore, as a lysate reagent, for example, a factor C of recombinant may be employed. After the addition of the detection reagent, incubation is performed at 37° C. for a predetermined time, and the light transmissivity of a reaction solution is measured. For the measurement of light transmissivity and the calculation of endotoxin, well-known techniques may be employed.

The use of the method for measuring endotoxin according to the present invention allows the influence of an interfering factor on the activity of endotoxin to be reduced. Therefore, it is possible to detect endotoxin with higher sensitivity and quantify with higher accuracy than was previously possible.

It should be noted that the method for measuring endotoxin according to the present invention may be executed by using the reagent for measuring endotoxin according to the present invention in combination with, for example, a commercial kit for endotoxin measurement.

### Examples

By showing the specific results of measurements by the above-mentioned reagent for measuring endotoxin and the above-mentioned method for measuring endotoxin according to the present invention, a more detailed description will be provided.

(Measurement of Endotoxin Recovery in the Presence of Interfering Factor)

As mentioned above, for example, iron sulfate, ciprofloxacin, and epirubicin have a direct interfering action on endotoxin. The effect of inhibiting the interfering action by the reagent for measuring endotoxin according to the present invention was examined.

Experimental conditions were as follows.

Endotoxin standard: E. coli UKT-B derived LPS

Addition concentration: 0.05 EU/mL

Measurement technique: turbidimetric technique using a lysate reagent

Lysate reagent: blood corpuscle extracts of horseshoe crab ES-II, freeze-dried

Measurement instrument: Toxinometer ET-6000/J

Calibration curve range: 0.1-0.025 EU/mL

An endotoxin solution was prepared using water for injection (Solution A). A 0.2 mM iron sulfate (FeSO₄) solution, a 2 mM aluminum chloride (AlCl₃) solution, a 2 mM gallium chloride (GaCl₃) solution, a 1 mM ciprofloxacin (CPFX) solution, a 1 mM minocycline (MINO) solution, a 0.05 mg/mL epirubicin solution, and a 1 mg/mL irinotecan solution, each containing 20 mM glucose-1-phosphate (G1P) or glucose-6-phosphate (G6P), were independently prepared (Solution B). Solution A and Solution B were mixed in equal proportions to prepare Solution C (the final concentration of G1P or G6P: 10 mM). Solution C was added into a lysate reagent and incubation was subsequently performed at 37° C., and light transmissivity was measured and the recovery of endotoxin was calculated.

As Comparative Examples, instead of G1P or G6P, water for injection (Water), a 20 mM glucose solution (Glu), or a 20 mM phosphate buffer (PB) was used for Solution B, and measurements were performed in the same manner.

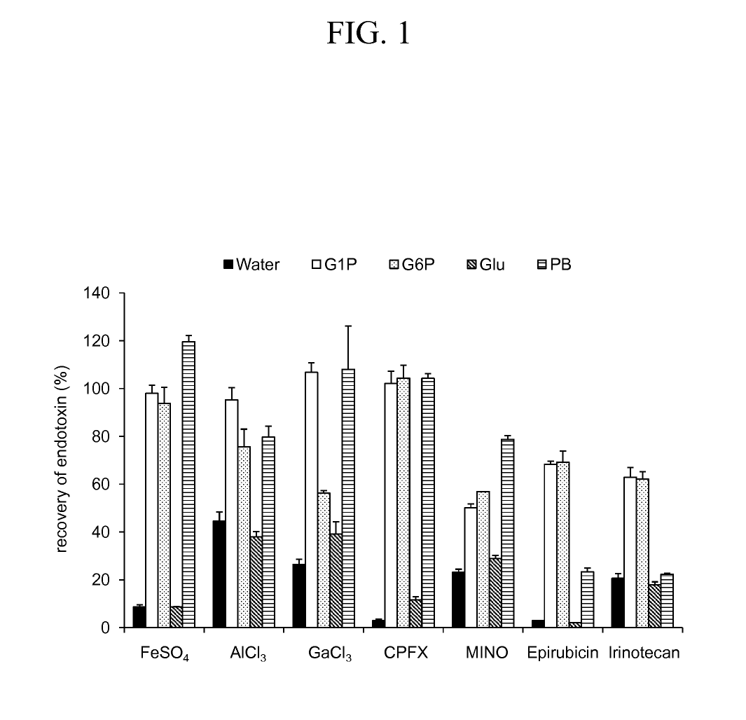

FIG. 1 shows the results of the measurements. FIG. 1 shows the recovery of endotoxin in the presence of an interfering factor in Examples in which the glucose-1-phosphate (G1P) or glucose-6-phosphate (G6P) containing reagent for measuring endotoxin according to the present invention was used and Comparative Examples in which water for injection, the glucose solution, or a phosphate buffer was used.

The glucose-1-phosphate (G1P) exerted inhibitory actions on most of the interfering factors used for the measurements at a level equal to or higher than the phosphate buffer. The phosphate buffer exerted an inhibitory effect on many of the interfering factors, but, in the samples containing epirubicin or irinotecan as an interfering factor, the phosphate buffer had a considerably low inhibitory effect on an interfering action. On the contrary, the glucose-1-phosphate (G1P) and glucose-6-phosphate (G6P) in Examples exerted an excellent inhibitory effect also on an interfering action of epirubicin or irinotecan. Glucose-6-phosphate (G6P) exerted a slightly lower inhibitory effect than glucose-1-phosphate, but, inhibited an interfering action of an interfering factor on endotoxin. On the other hand, glucose, which is similar to G1P and G6P, did not inhibit an interfering action of an interfering factor on endotoxin. From this result, it is inferred that a phosphate group added to a sugar competes with a phosphate group of Lipid A for an interfering factor, thereby directly inhibiting the interference.

In Examples, the use of glucose-1-phosphate or glucose-6-phosphate allowed neutralization effects equal to or higher than the use of the buffer to show up. In particular, in an anticancer drug, which has high bioactivity, the use of glucose-1-phosphate or glucose-6-phosphate brought a neutralization effect higher than the use of the buffer. This result suggest that, through the use of glucose-1-phosphate or glucose-6-phosphate, a higher neutralization effect even on a substance which cannot be sufficiently neutralized by the buffer can be expected. Furthermore, because of the use of glucose-1-phosphate or glucose-6-phosphate, nonspecific turbidity which is sometimes caused when the buffer is used does not need to be considered, and application to a wider variety of substances is made possible accordingly.

(Measurement of Endotoxin in Plasma)

As mentioned above, when a measurement of blood endotoxin concentration is performed, endotoxin specifically or nonspecifically binds to proteins, and interference is caused accordingly. The effect of inhibiting an interfering action by the reagent for measuring endotoxin according to the present invention was examined.

(Examination Using Rat Plasma)

From the whole blood of a rat, plasma was separated by centrifugation, an endotoxin standard was added thereto, and a measurement of blood endotoxin concentration was performed. Experimental conditions were as follows.

Animal subject: SD rat, male, 6 ages in week (200 to 230 g in weight)

Blood collection: heparin blood collection and icing.

Plasma separation: 700×g at 4 C.° for 3 minutes

Endotoxin standard: E. coli, UKT-B derived LPS

Addition concentration: 0.0025 EU/mL

Measurement technique: turbidimetric technique using a lysate reagent
Lysate reagent: Limulus ES-II, Single Test wako
Measuring equipment: Toxinometer, ET-6000/J
Minimum limit of determination: 0.000625 EU/mL

To prepare a sample, endotoxin was added to plasma so as to attain an endotoxin concentration of 0.025 EU/mL, and as Examples, the resulting plasma was diluted 10 times with 2.5 mM glucose-1-phosphate (G1P) or 2.5 mM glucose-6-phosphate (G6P). As Comparative Examples, the resulting plasma was diluted with water for injection (Water), a 20 mM glucose solution (Glu), or a 20 mM phosphate buffer (PB), respectively, instead of G1P or G6P, in the same manner. The diluted plasma was heated at 75° C. for 10 minutes. After the plasma was added to a lysate reagent, incubation was performed at 37° C., and light transmissivity was measured.

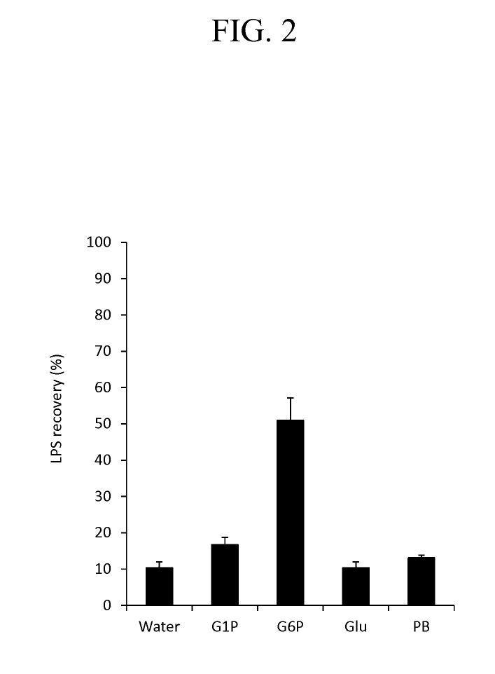

FIG. 2 shows the results of the measurements. In the case of the 10-fold dilution with water for injection (Water), the recovery of endotoxin was approximately 10%, on the other hand, the use of 2.5 mM glucose-1-phosphate (G1P) or 2.5 mM glucose-6-phosphate (G6P) brought an endotoxin recovery of not less than 15%. In particular, in the case of the addition of G6P, a high recovery of a little over 50% was achieved. On the contrary, the use of a phosphate buffer resulted in little improving effect on endotoxin recovery. This result suggests that the reagent for measuring endotoxin according to the present invention inhibits an interfering action of proteins in plasma.

It was proved that, compared with a prior art in which 10-fold dilution with water for injection and subsequent heating at 75° C. for 10 minute are performed, in the reagent for measuring endotoxin according to the present invention, glucose-1-phosphate or glucose-6-phosphate is used, whereby an interfering action of proteins in rat plasma is neutralized, and the recovery of endotoxin is improved accordingly.

(Examination Using Human Plasma)

Using human plasma, the same examination was performed. From whole human blood, plasma was separated by centrifugation. Endotoxin was added to the plasma so as to attain an endotoxin concentration of 0.025 EU/mL, and as Examples, the resulting plasma was diluted 10 times with 2.5 mM glucose-1-phosphate (G1P) or 2.5 mM glucose-6-phosphate (G6P). As Comparative Examples, the resulting plasma was diluted with water for injection (Water), a 2.5 mM glucose solution (Glu), or a 2.5 mM phosphate buffer (PB), instead of G1P or G6P, in the same manner. The diluted plasma was heated at 70° C. for 10 minutes. After the plasma was added to a lysate reagent, incubation was performed at 37° C., and light transmissivity was measured.

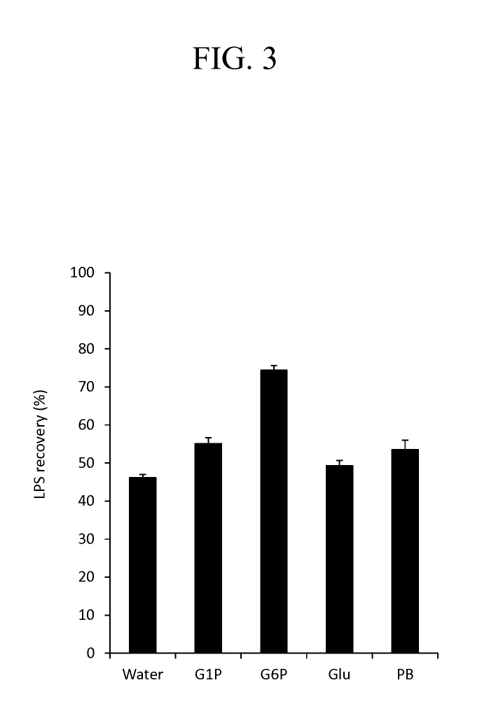

FIG. 3 shows the results of the measurements. In the case of the 10-fold dilution with water for injection, the recovery of endotoxin was approximately 45%, on the other hand, the use of 2.5 mM glucose-1-phosphate (G1P) or 2.5 mM glucose-6-phosphate (G6P) brought an endotoxin recovery of 55% or more. In particular, in the case of the addition of G6P, a high recovery of 75% was achieved. On the contrary, although the use of a phosphate buffer also led to a slight improvement on endotoxin recovery, the endotoxin recovery was lower than those in the cases of using G1P and G6P. This result suggests that the reagent for measuring endotoxin according to the present invention inhibits an interfering action of proteins in plasma.

It was proved that, compared with a prior art in which 10-fold dilution with water for injection and subsequent heating at 70° C. for 10 minute are performed, in the reagent for measuring endotoxin according to the present invention, glucose-1-phosphate or glucose-6-phosphate is used, whereby an interfering action of proteins in human plasma is neutralized, and a high endotoxin recovery is achieved accordingly. This result suggest that the use of the reagent for measuring endotoxin according to the present invention makes it possible to measure endotoxin in plasma with high accuracy, and the prevention of a false-negative diagnosis of sepsis can be expected.

(In Vivo Measurement of Endotoxin)

As mentioned above, in the in vitro measurement of endotoxin, the reagent for measuring endotoxin according to the present invention significantly inhibited an interfering action of an interfering factor on endotoxin. Next, an inhibitory effect on interfering action in the in vivo measurement of endotoxin using a rat was examined.

First, sepsis model rats were produced. Experimental conditions were as follows.

Animal subject: SD rat, male, 6 ages in week (200 to 230 g in weight)

Sepsis model: cecal ligation and puncture (ligate an end of the cecum on the side of the ileum, and make punctures at 2 points with an 18G injection needle)

Blood collection: heparin blood collection performed 4 hours after treatment, and icing

Plasma separation: 700×g at 4° C. for 3 minutes

Dilution and heating: Separated plasma was diluted 10 times with water for injection or 2.5 mM glucose-6-phosphate (G6P), and then heated for 10 minutes.

Measurement technique: turbidimetric technique using a lysate reagent
Lysate reagent: Limulus ES-II, Single Test wako
Measurement instrument: Toxinometer ET-6000/J
Endotoxin for calibration curves: E. coli, UKT-B origin LPS
Minimum limit of determination: 0.000625 EU/mL

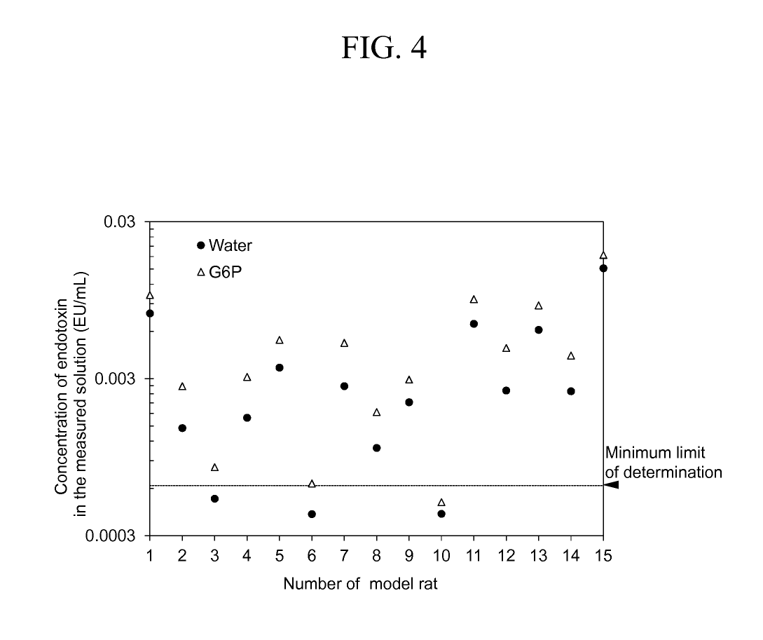

From the whole blood of the sepsis model rats, plasma was separated by centrifugation. The separated plasma was diluted 10 times with water for injection (Water) or 2.5 mM glucose-6-phosphate (G6P), and then heated at 75° C. for 10 minutes, and endotoxin in the rat plasma was measured. FIG. 4 shows the measurement results of endotoxin in the plasma of the sepsis model rats. The result of measurements of the 15 sepsis model rats were that the use of the glucose-6-phosphate (G6P) containing reagent for measuring endotoxin according to the present invention led to a high concentration of endotoxin in the plasma of any of the sepsis model rats. This result suggests that the use of the glucose-6-phosphate (G6P) containing reagent for measuring endotoxin according to the present invention makes it possible to reduce the influence of an interfering factor in the sample and thereby to measure endotoxin with higher sensitivity even in vivo.

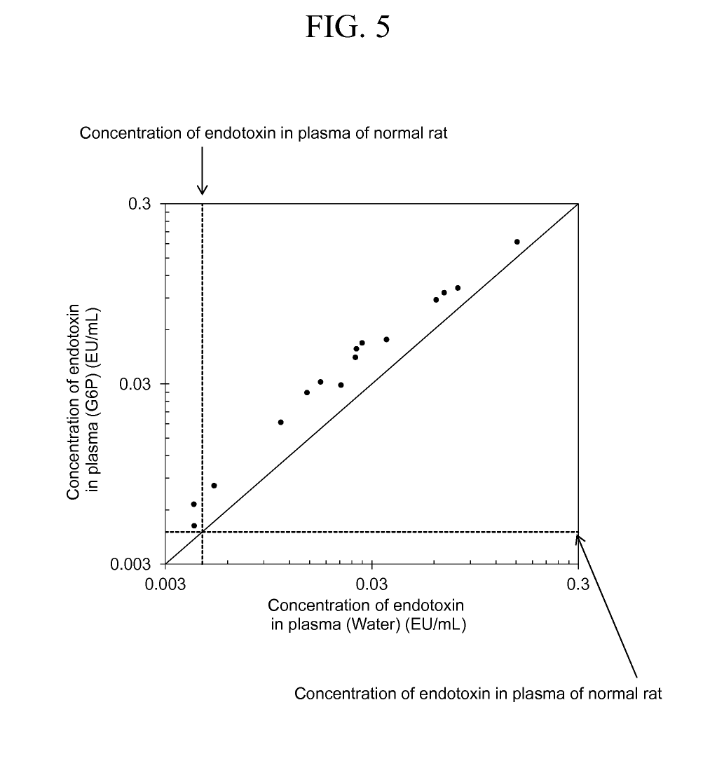

FIG. 5 shows the result of a comparison of blood endotoxin concentration between the case where the concentration of endotoxin in plasma was measured using water for injection and the case where the concentration thereof was measured using the reagent for measuring endotoxin according to the present invention. Also from the result shown in FIG. 5, it is clear that, in all the cases, the use of the reagent for measuring endotoxin according to the present invention brought higher blood endotoxin concentrations than the dilution with water for injection. Furthermore, as for two cases (shown at the lower left part of the graph) in FIG. 5, when dilution was performed with water for injection (Water), the concentrations of endotoxin in plasma were lower than that of a normal rat, but on the contrary, when the glucose-6-phosphate (G6P) containing reagent for measuring endotoxin according to the present invention was used, the concentrations of endotoxin in plasma were higher than that of a normal rat, and hence, it can be expected that the influence of an interfering factor in a sample is reduced even in vivo and the risk of a false negative diagnosis is reduced accordingly.

The present invention provides a reagent for measuring endotoxin and a method for measuring endotoxin which reduce the influence of an interfering factor. The reagent for measuring endotoxin and the method for measuring endotoxin according to the present invention can be made use of for measuring endotoxin in pharmaceuticals and endotoxin in blood. Furthermore, the reagent for measuring endotoxin and the method for measuring endotoxin according to the present invention can be made use of for measuring endotoxin in experimental materials, such as a chemical, a reagent, and a medium.

1. A method for measuring endotoxin, comprising:
adding glucose-1-phosphate or glucose-6-phosphate to a sample, and
adding a detection reagent to the sample.

2. The method for measuring endotoxin according to claim 1, wherein the glucose-1-phosphate or the glucose-6-phosphate is added in a form of a solution to the sample.

3. The method for measuring endotoxin according to claim 2, wherein the sample is a non-blood sample.

4. The method for measuring endotoxin according to claim 3, wherein the non-blood sample is a pharmaceutical.

5. The method for measuring endotoxin according to claim 4, wherein the pharmaceutical is a pharmaceutical preparation, a drug substance, or an inactive additive.

6. The method for measuring endotoxin according to claim 3, wherein the non-blood sample is a chemical, a reagent, or a medium.

7. The method for measuring endotoxin according to claim 2, wherein a solution containing the glucose-1-phosphate or the glucose-6-phosphate is prepared so as to have a concentration of the glucose-1-phosphate or the glucose-6-phosphate of 1 mM or more and 25 mM or less.

8. The method for measuring endotoxin according to claim 2, wherein the sample is a blood sample.

9. The method for measuring endotoxin according to claim 8, wherein the blood sample is plasma.

10. The method for measuring endotoxin according to claim 9, wherein the glucose-1-phosphate or the glucose-6-phosphate is added to the plasma, the plasma is heat-treated, and a detection reagent is added to heat-treated plasma.

11. The method for measuring endotoxin according to claim 8, wherein a solution containing the glucose-1-phosphate or the glucose-6-phosphate has a concentration of the glucose-1-phosphate or the glucose-6-phosphate of 1 mM or more and 5 mM or less.

12. A reagent for measuring endotoxin, comprising glucose-1-phosphate or glucose-6-phosphate.

13. The reagent for measuring endotoxin according to claim 12, wherein the reagent is a solution containing the glucose-1-phosphate or the glucose-6-phosphate.

14. The reagent for measuring endotoxin according to claim 13, wherein the solution has a concentration of the glucose-1-phosphate or the glucose-6-phosphate of 1 mM or more and 25 mM or less.

15. The reagent for measuring endotoxin according to claim 13, wherein the solution has a concentration of the glucose-1-phosphate or the glucose-6-phosphate of 1 mM or more and 5 mM or less.
